(19) United States
(12) Patent Application Publication
(10) Pub. No.: US 2004/0161810 A1
(43) Pub. Date: Aug. 19, 2004

(54) Monoclonal antibodies specific for latent antithrombin III and the use thereof

(76) Inventors: Juergen Roemisch, Vosendorf (DE); Wiegand Lang, Coelbe (DE); Annette Feussner, Marburg (DE); Harald Stauss, Dautphetal (DE)

Correspondence Address: Finnegan, Henderson, Farabow,, Garrett & Dunner, L.L.P., 1300 I Street, N.W., Washington, DC, 20005-3315, US

(21) Appl. No.: 10/367,747

(22) Filed: Feb. 19, 2003

(51) Int. Cl. G01N 33/53

(52) U.S. Cl. 435/7.92

(57) ABSTRACT

The present invention provides antibodies that are specific for the latent or locked form of antithrombin III (AT III). The antibodies have a specificity for latent antithrombin III (1-AT III) that is higher than the specificity for natural AT III. The invention also provides methods of making and 1-AT III. It further provides methods of detecting and quantitating 1-AT III in biological samples.

This invention relates to monoclonal antibodies specific for latent antithrombin III (AT III), their production, and the use thereof in therapy and diagnosis of diseases and the use thereof in the preparation of latent AT III.

AT III is a form of antithrombin (AT), which is one of the serine proteinase inhibitors (SERPIN) and occurs in human plasma on average in a concentration of 150 mg/l. AT is an inhibitor essentially of thrombin, but it also inhibits the activity of other activated coagulation factors such as FXa, FIXa etc., and is thus the most important regulator of hemostasis. Its importance is made particularly clear by the fact that the risk of mortality increases drastically when the AT level in plasma falls below 70% of normal. This is why AT concentrates are produced from plasma and are used for replacement in congenital or acquired deficiency states. The anticoagulant properties of AT are distinctly increased by heparin and other glycosaminoglycans. By contrast, recent investigations have shown that, in the presence of even small amounts of heparin, AT loses its anti-inflammatory properties, such as the reduction of cellular activations induced by pro-inflammatory substances, and the expression of, for example, cytokines and thromboplastin. This result can be attributed to the fact that AT can interact with cells, which interaction in turn is based on the accessibility of the so-called heparin binding site (HBS). AT is able, with the aid of the latter, to interact with cellular surface structures and receptors, and thus mediate anti-inflammatory effects. However, the AT binding site blocked by soluble heparin prevents such an interaction with the cells.

As a typical SERPIN, AT (including the AT III isoform) has a so-called reactive site loop in the vicinity of the carboxyl terminus, where the essential interaction of the inhibitor with its target proteases takes place. This involves reaction of the active site of the protease with particular regions of this loop, which eventually leads to cleavage thereof, resulting in a quasi-covalent, very stable protease-inhibitor complex. AT is thus a so-called suicide inhibitor because, after this cleavage, it is no longer able to inhibit further activated protease molecules.

Other proteases, such as, for example, elastase, cleave at an adjacent site in this AT loop, which leads to inactivation of AT but not to an inhibitor complex with this protease. The structural result of this cleavage in AT is a drastic change in conformation of the inhibitor, with the loop (C terminus) swinging into a “molecule recess” and being converted into a form which is stable but has no protease inhibitory activity, and which is referred to as “locked”. This is also frequently described as a “latent” conformation because, for example, it has been known for a long time for PAI-1 (plasminogen activator inhibitor 1) and can be converted back into the potent inhibitor conformation by appropriate treatment. This refolding is, according to the current state of knowledge, not possible with AT. That is, the so-called latent form is really locked and thus remains useless as protease inhibitor.

Besides the proteolytic inactivation and subsequent conversion into the latent/locked form of AT, input of energy in the form, for example, of heating can also lead to misfolding in the above-mentioned sense. This entails the entire (uncleaved) C terminus swinging into the above-mentioned recess and leading to a state which is structurally very similar to that resulting from cleavage. Accordingly, the conformation of AT produced by a rise in temperature is not a protease inhibitor either. It is also true of these latent/locked configurations that, in the drastic restructuring of the molecule, the changes in the regions of the molecule necessary for heparin binding of AT are such that these ATs lose affinity for glycosaminoglycans such as heparin, and thus association with the latter is restricted. Correspondingly, latent/locked AT (1-AT) can be separated relatively easily from active AT by adsorption on immobilized heparin.

Until recently, it was not possible to assign a biological function to 1-AT; rather it was regarded as an (artificial) end product of a reaction which, as an inactive product, is eliminated rapidly from the body. A recent publication by O'Reilly et al. showed that 1-AT is a potent inhibitor of tumor angiogenesis. Both in cellular in vitro models and in tumor mouse models, administration of 1-AT led to a significant reduction in neoangiogenesis (and endothelial cell proliferation) and thus in tumor growth. Since the nutrient and oxygen supply is an important precondition for rapid growth of the tumor, the reduction or inhibition of blood vessels supplying the tumor leads to cessation of growth of the tumor. This realization opens up the clinical possibility of further antitumor therapies. In favorable cases, suppressing the supply to tumors may also lead to regression of the tumor. This realization opens up the possibility of effective tumor therapy with 1-AT.

Detection of AT generally in body fluids has been established for some time. Both antigen quantification systems in the form of immunoassays, and the determination of inhibitory capacities mediated by active AT are available. However, it is not presently possible to detect or quantify 1-AT satisfactorily in fluids or tissues. Only recently have Carrell et al. reported a detection system based on gel electrophoresis and differentiation of the AT/1-AT forms. The validity of this assay is as yet unproven. Even if this system is suitable for differentiating the two conformations, this differentiation is not suitable either for quantitative evaluation or for detecting 1-AT, for example, in histological investigations.

A further possibility for differentiation is provided in the form of two-dimensional immunoelectrophoresis. This entails AT being subjected to a first electrophoresis in the presence of heparin. Whereas the active AT molecules which bind heparin migrate rapidly, the slowly migrating 1-AT molecules are separated out. A further electrophoresis (rotated by 90° relative to the first) into an agarose gel containing polyclonal antibodies against complete AT leads to precipitation of AT-antibody complexes, which can be stained. The AT or 1-AT regions revealed as precipitation peaks are quantified as areas under the curves. This method is reliable but insensitive. It is possible to differentiate 1-AT only as a proportion of >2% of total AT, which would correspond to a sensitivity of about 3 μg/ml in plasma samples. However, the described parameters apply to the pure AT system. If plasma is used, however, the sensitivity is further reduced because the protein concentration leads to distortion of the bands and impedes evaluation. In addition, this method is useless for histological purposes. Since 1-AT is being considered for the treatment of cancer, monitoring of administered 1-AT is necessary, for example to determine in vivo half-lives or tissue distributions. Since AT is detected in plasma and many tissues, at present differential examination of 1-AT is not possible.

Because of the lack of assay systems, it is not known what concentrations of 1-AT are produced, and by which cells or organs, during which physiological and pathophysiological reactions. In accordance with the existing theories that 1-AT might be an endogenous agent for defending against tumors, there is also great interest in detecting it in tissues (histology), in its interaction with cells (for example, FACS analyses) or else in its detection (e.g., Western blotting) and quantification (e.g., immunoassays such as ELISA) in body fluids. Small concentrations of 1-AT might also be generated through an increase in body temperature, for example through fever, and detection thereof may be of analytical interest.

Accordingly, antibodies able to differentiate 1-AT from active AT would be of great interest. We have now identified, surprisingly, through immunization of mice with 1-AT, monoclonal antibodies which recognize 1-AT specifically and can thus be used for the abovementioned methods. Sixteen of 255 tested clones distinguish between AT and 1-AT. Although all of these antibodies are capable of differentiation, 3 of them are exceptionally well-suited for the above-mentioned applications.

Since, according to the current state of knowledge, the (patho)physiological concentrations of 1-AT to be expected are only very low (e.g., in the nanogram or lower microgram range per milliliter of plasma), the specificity of a suitable antibody must be very high, especially in an environment such as plasma, in which on average 150 μg/ml total AT are present. For specific quantitative determination of, for example 100 ng of 1-AT/ml, it is preferable, and perhaps necessary, for the factor for differentiation from AT to be about or greater than 1,000.

Accordingly, the present invention provides a monoclonal antibody or a fragment of a monoclonal antibody that specifically reacts with an epitope specific for latent/locked antithrombin III (1-AT III). The antibody or fragment of the invention can be any antibody that shows a greater specificity for 1-AT III than natural AT III. Included among the antibodies of the invention are polyclonal and monoclonal antibodies.

The antibodies or fragments can have a specificity of 100-fold or more greater for 1-AT III than natural AT III. Likewise, they can have a specificity of 250-fold or more greater, 500-fold more greater, 750-fold more or greater, or 900-fold more or greater for 1-AT III than natural AT III. As used herein, levels of specificity are given with the understanding that the precision of measurement is limited by current techniques. Thus, the numbers provided are not to be strictly limiting of the level of specificity, but are to encompass all levels that cannot be clearly differentiated from the stated level using one or more techniques typically used in the art.

In embodiments, the antibody is a monoclonal antibody or fragment that has a specificity for 1-AT III that is 1,000-fold or more greater than its specificity for active AT III. For example, the monoclonal antibody or fragment can have a specificity for 1-AT III that is more than 1,000-fold greater than its specificity for active AT III. In addition, it can have a specificity for 1-AT III that is 5,000-fold or more greater than its specificity for active AT III, or a specificity for 1-AT III that is 10,000-fold or more greater than its specificity for active AT III. In sum, the present invention provides, among other things, monoclonal antibodies and fragments that can have a specificity for 1-AT III that is between 1,000- and 10,000-fold greater, or more, than its specificity for active AT III. As detailed in Example 1, antibodies according to the present invention can show a factor for differentiation of >10,000 with high sensitivity, and are therefore outstandingly suitable for specific detection and quantification of 1-AT III.

The procedures disclosed in the present application make it possible for those of skill in the art to identify, without undue experimentation, a monoclonal antibody or fragment of a monoclonal antibody that has these levels of specificity.

Latent AT-specific monoclonal antibodies can be obtained and identified using methods familiar to the skilled worker and therefore require only a short description. The following procedure is provided solely as an exemplary disclosure of the invention, and is directly relevant to the non-limiting disclosure of Example 1, below.

Monoclonal antibodies were produced by immunizing mice with 1-AT. The spleen cells of an immunized mouse were fused with the murine myeloma cell line SP2/0-Ag14 using PEG 1500 as fusion reagent. The cells were distributed on 24-well culture plates. The medium used was Dulbecco's modified Eagle's medium with 10% fetal calf serum. After 10-14 days, the growing cell clones were transferred into the wells of a 48-well culture plate and coded. The culture supernatant was taken from 2,400 grown clones and tested by ELISA for binding to 1-AT. In total, 255 clones were then investigated for cross-reactivity with normal AT, alpha-AT, beta-AT, and goat AT. Sixteen clones showed binding. That is, they were able to distinguish between AT and 1-AT. The specificity of the antibodies produced by these hybridoma cells was confirmed in a BIACORE (biomolecular interaction analysis).

Thus, in an embodiment, the present invention provides a monoclonal antibody or fragment of a monoclonal antibody, which is obtained by: i) immunizing mice with I-AT III; ii) isolating spleen cells from the immunized mice; iii) fusing the spleen cells with SP2/0-Ag 14 murine cells to make at least one hybridoma cell; iv) growing the resulting hybridoma cells; v) identifying clones producing a monoclonal antibody or fragment according to the invention; and vi) using the clones to produce an antibody or fragment according to the invention.

As described above, the antibodies and fragments of the present invention are suitable for a number of analytical assays known to those of skill in the art. It is moreover possible to use the antibodies alone or in combination. Likewise, fragments, such as the corresponding F(ab′) and F(ab′)2 fragments, can be used according to the invention. For example, binding of the antibodies or of the fragments can be followed through direct labeling thereof using, for example, markers, such as fluorescent or coloring molecules, which are familiar to the skilled worker, as are coupling of enzymes and substrate conversion thereby, etc.

In addition, detection is also possible by using labeled anti-mouse antibodies, or other labels known to those of skill in the art. In an exemplary embodiment, the 1-AT-specific antibodies and fragments can be employed for so-called “capture” in the case of an immunoassay. However, in the labeled state, they can also serve as detectors if, for example, there is initial binding of AT/1-AT mixtures (for example by antibodies which are unable to distinguish between the two conformations). Many other labels, detection systems, and reagents are known to those of skill in the art. All suitable labels, systems, and reagents may be used according to the present invention.

Thus, the invention provides a method of detecting 1-AT III in a body fluid, tissue, or 1-AT III-containing preparation. In an embodiment, the method comprises: i) contacting the body fluid, tissue, or 1-AT III-containing preparation with a monoclonal antibody or fragment according to the invention; and ii) detecting the amount of bound monoclonal antibody or fragment, the amount of free monoclonal antibody or fragment, or both. The method can further comprise determining the quantity of bound monoclonal antibody or fragment, the quantity of unbound monoclonal antibody or fragment, or both. Thus, the method can be both qualitative and quantitative, and can be used to evaluate the presence or likelihood of a tumor state, and to monitor the in vivo status of a therapeutic regimen that relies, at least in part, on knowledge of the plasma or tissue level of 1-AT III, or the ratio of natural AT III to 1-AT III.

Besides the possibility of using the 1-AT-specific antibodies and fragments of the invention for analysis, they can also be used for preparing 1-AT. The immunoadsorbent obtained by immobilization on matrices familiar to the skilled worker, such as SEPHAROSE® or FRACTOGEL®, can be incubated with 1-AT-containing solutions, the solution containing the other proteins (including active AT) can be removed, and subsequently the 1-AT can be obtained by elution. This 1-AT can then be used for analytical or therapeutic purposes. Conversely, it is also possible to free a solution of 1-AT. This is desirable, for example, if it is wished to use only active AT, for example in an AT concentrate administered for anticoagulant and anti-inflammatory prophylaxis and therapy.

Thus, the present invention provides a method of producing 1-AT III from a body fluid, tissue, or 1-AT III-containing preparation. The method comprises: i) contacting the body fluid, tissue, or 1-AT III-containing preparation with the monoclonal antibody or fragment of the invention under conditions where 1-AT III that is present in the fluid, tissue, or preparation binds to the monoclonal antibody or fragment; and ii) releasing the bound 1-AT III. The method of producing 1-AT III can, of course, include isolating, either fully or partially, the bound 1-AT III from some or all of the other substances present, to produce a sample of purified 1-AT III or a sample that is free, essentially free, or free to a desired extent of 1-AT III. By essentially free, it is meant that the sample contains only that amount of 1-AT III that cannot be practically removed using standard biological, biochemical, and chemical methods.

At present, nothing is known about the half-life (t_{1/2}) of 1-AT in body fluids and tissues. However, an antibody that reacts specifically with 1-AT might serve to prolong the t_{1/2} because it is known that antibodies have a t_{1/2} (in blood) of several weeks. Accordingly, multiple administration of potentially “short-lived” 1-AT can be limited to a relatively few doses to be administered at relatively long time intervals.

The invention is further described in detail by the following Example, which is to be considered exemplary of the invention, and not to be construed as limiting the invention in any way.

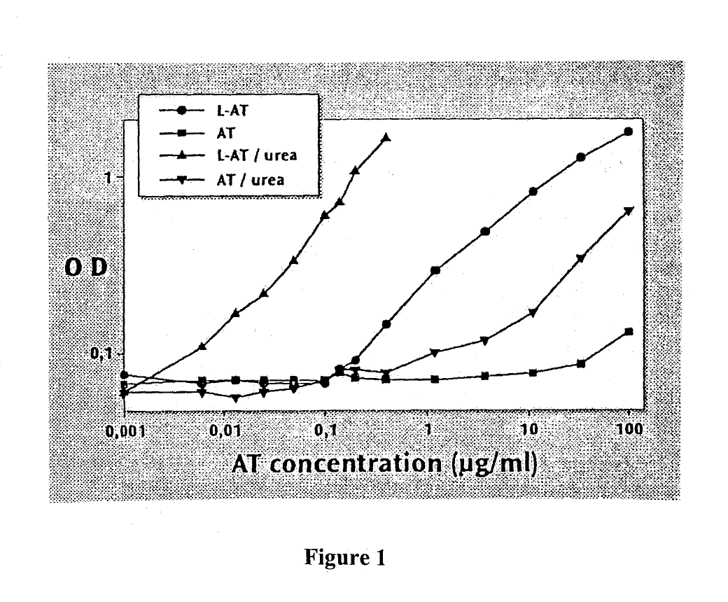

FIG. 1, which depicts results obtained in Example 1, shows the specificity of MAb-804 (a monoclonal antibody according to the present invention) for 1-AT III over natural AT III. FIG. 1 is a plot of the results of an ELISA, showing the extinction (O.D.) vs. concentration of natural AT III and 1-AT III, with or without treatment with urea.

### Example 1

The specificity of antibodies for 1-AT was demonstrated by carrying out an immunoassay in the form of an ELISA. For this purpose, 1-AT was prepared from the antithrombin III concentrate KYBERNIN®P (Aventis Behring, Marburg, Germany) by adsorption of active AT on immobilized heparin. Accordingly, the 1-AT is obtained in the adsorbent supernatant, and the active AT is obtained in the eluate, as can also be demonstrated using the heparin cofactor assay familiar to the skilled worker.

A 96-well plate was coated with 10 μg/ml of polyclonal (rabbit) antibodies against antithrombin, which bind both AT and 1-AT. The wells were then incubated with increasing concentrations of AT or 1-AT, washed, and subsequently incubated with MAb-804. Removal of the supernatant was followed by detection using a labeled anti-mouse monoclonal antibody and measurement of the extinction. The resulting extinctions were plotted against the concentrations of AT and 1-AT, as shown in FIG. 1.

Whereas no significant increase in the extinction was measurable in AT even at high concentrations, the extinction in the mixtures incubated with 1-AT increased markedly and reached, in a concentration-dependent manner, extinctions of up to 3.0. As shown in FIG. 1, a factor for differentiation between AT and 1-AT of >10,000 was found, with high sensitivity of 1-AT detection. This means that quantification of 1-AT will be possible even in solutions in which high concentrations of AT are present.

What is claimed is:

1. A monoclonal antibody or a fragment of a monoclonal antibody that specifically reacts with an epitope specific for latent/locked antithrombin III (1-AT III).

2. The monoclonal antibody or fragment of claim 1, wherein the specificity of the antibody or fragment for 1-AT III is 1,000-fold greater than its specificity for active AT III.

3. The monoclonal antibody or fragment of claim 1, wherein the specificity of the antibody or fragment for 1-AT III is more than 1,000-fold greater than its specificity for active AT III.

4. The monoclonal antibody or fragment of claim 3, wherein the specificity of the antibody or fragment for 1-AT III is 5,000-fold greater than its specificity for active AT III.

5. The monoclonal antibody or fragment of claim 3, wherein the specificity of the antibody or fragment for 1-AT III is more than 5,000-fold greater than its specificity for active AT III.

6. The monoclonal antibody or fragment of claim 3, wherein the specificity of the antibody or fragment for 1-AT III is 10,000-fold greater than its specificity for active AT III.

7. The monoclonal antibody or fragment of claim 3, wherein the specificity of the antibody or fragment for 1-AT III is more than 10,000-fold greater than its specificity for active AT III.

8. The monoclonal antibody or fragment of claim 3, wherein the specificity of the antibody or fragment for 1-AT III is between 1,000- and 10,000-fold greater than its specificity for active AT III.

9. The monoclonal antibody or fragment of claim 1, which is obtained by
i) immunizing mice with 1-AT III;
ii) isolating spleen cells from the immunized mice;
iii) fusing the spleen cells with SP2/0-Ag 14 murine cells to make at least one hybridoma cell;
iv) growing the resulting hybridoma cells;
v) identifying clones producing the monoclonal antibody or fragment of claim 1; and
vi) using the clones to produce the antibody or fragment of claim 1.

10. The monoclonal antibody or fragment of claim 1, wherein the antibody or fragment comprises a detectable label.

11. A method of detecting 1-AT III in a body fluid, tissue, or 1-AT III-containing preparation, said method comprising:
i) contacting the body fluid, tissue, or 1-AT III-containing preparation with the monoclonal antibody or fragment of claim 1; and
ii) detecting the amount of bound monoclonal antibody or fragment of claim 1, the amount of free monoclonal antibody or fragment of claim 1, or both.

12. The method of claim 11, further comprising determining the quantity of bound monoclonal antibody or fragment, the quantity of unbound monoclonal antibody or fragment, or both.

13. The method of claim 12, wherein the quantity or quantities are determined by ELISA or Western blotting.

14. The method of claim 11, wherein the method further comprises determining the ½life of 1-AT III in the body fluid, tissue, or 1-AT III-containing preparation.

15. The method of claim 11, wherein the method is a method of detecting 1-AT III in a histological sample.

16. A method of producing 1-AT III from a body fluid, tissue, or 1-AT III-containing preparation, said method comprising:
i) contacting the body fluid, tissue, or 1-AT III-containing preparation with the monoclonal antibody or fragment of claim 1 under conditions where 1-AT III that is present in the fluid, tissue, or preparation binds to the monoclonal antibody or fragment; and
ii) releasing the bound 1-AT III.

17. The method of claim 16, wherein the bound 1-AT III is isolated from some or all of the other substances present.
